(19) United States
(12) Patent Application Publication
(10) Pub. No.: US 2021/0015711 A1
(43) Pub. Date: Jan. 21, 2021

(54) MEDICINE VERIFICATION DEVICE AND MEDICINE VERIFICATION METHOD

(71) Applicant: FUJIFILM Toyama Chemical Co., Ltd., Tokyo (JP)

(72) Inventor: Kazuchika IWAMI, Ashigarakami-gun (JP)

(73) Assignee: FUJIFILM Toyama Chemical Co., Ltd., Tokyo (JP)

(21) Appl. No.: 17/039,464

(22) Filed: Sep. 30, 2020

Related U.S. Application Data
(63) Continuation of application No. PCT/JP2019/008981, filed on Mar. 7, 2019.

(30) Foreign Application Priority Data
Apr. 3, 2018 (JP) 2018-071901
Nov. 22, 2018 (JP) 2018-218896

(51) Int. Cl. A61J 7/00; G16H 20/10; G16H 30/00; B65B 5/10

(52) U.S. Cl. CPC A61J 7/0084; B65B 5/103; G16H 30/00; G16H 20/10

(57) ABSTRACT

Provided are a medicine verification device and a method capable of performing accurate verification of the type of medicines even in a case where the sharpness of the image differs between a master image and a captured image of the verification target medicine. The type of the verification target medicine packed in a packaging material is verified by using the master image and the captured image of the verification target medicine, and then characteristic information indicating an optical characteristic of the packaging material is acquired. When the optical characteristic indicated by the characteristic information is an optical characteristic that decreases the sharpness of the captured image of the verification target medicine to be lower than that of the master image, correction processing is executed and the type of the verification target medicine is verified using the master image in which the sharpness has been corrected by the correction processing.

### CROSS-REFERENCE TO RELATED APPLICATIONS

This application is a Continuation of PCT International Application No. PCT/JP2019/008981 filed on Mar. 7, 2019, which claims priority under 35 U.S.C. § 119(a) to Japanese Patent Application No. 2018-071901 filed on Apr. 3, 2018 and Japanese Patent Application No. 2018-218896 filed on Nov. 22, 2018. Each of the above applications is hereby expressly incorporated by reference, in its entirety, into the present application.

### BACKGROUND OF THE INVENTION

The present invention relates to a medicine verification device and a medicine verification method, and more particularly relates to a medicine verification device and a medicine verification method adapted to verify the type of verification target medicine using a master image registered in association with the type of medicine and a captured image of the verification target medicine.

As an already known technique, an automatic inspection has been performed by a machine or the like as to whether a medicine is correctly packaged in a packaging bag such as a sachet sheet as instructed by a prescription. Such an automatic inspection device (hereinafter, referred to as a medicine verification device) captures an image of a medicine packed in a packaging bag, for example, within the device. Subsequently, the device verifies the type and number of the medicines appearing in the captured image.

Furthermore, for example, a verification method described in JP 2005-249615 A (in particular, refer to claim 1 and paragraphs 0021 to 0022 of JP 2005-249615 A) is used to compare a captured image of a workpiece as a verification target and an image of a non-defective workpiece being a reference image (hereinafter, a master image) to verify whether the workpiece as a verification target matches the non-defective workpiece.

Furthermore, according to the verification method described in JP 2005-249615 A, the master image is updated with the captured image of the verification target workpiece determined to match the non-defective workpiece in the verification, as a new master image. With this configuration, image comparison is always performed with the latest captured image of the workpiece verified to match the non-defective workpiece, as the master image. As a result, for example, even with a difference in the illuminance of the illumination at individual timings of image capturing or with a gradual change in the illuminance, it is possible to continuously perform verification without processes of adjusting the illumination, changing the threshold, re-registering the master image, or the like. That is, according to the verification method described in JP 2005-249615 A, it is possible to perform verification appropriately without being influenced by the environmental change, as compared with the case of performing fixed registration of a master image.

### SUMMARY OF THE INVENTION

In capturing an image of a verification target medicine, the medicine is imaged in a state of being packed in a packaging bag having light transmissivity (that is, through the packaging bag). For this reason, the sharpness of the captured image changes in accordance with the light transmissivity, light scattering and the like of the packaging bag. Here, a difference between the optical characteristic of the packaging bag packing the verification target medicine and the optical characteristic of the packaging bag packing the medicine appearing in the master image would lead to a difference in the sharpness of the medicine in both images, and this difference in sharpness might influence the verification accuracy.

The present invention has been made in view of the above circumstances and aims to provide a medicine verification device and a medicine verification method capable of performing accurate verification of the type and the like of medicine even in a case where the sharpness of the image differs between the master image and the captured image of the verification target medicine.

In order to achieve the above object, the medicine verification device of the present invention includes: an image capturing unit configured to capture an image of a verification target medicine packed in a packaging material having light transmissivity; a verification unit configured to verify a type of the verification target medicine using a master image registered in association with the type of medicine and the image of the verification target medicine captured by the image capturing unit; a characteristic information acquisition unit configured to acquire characteristic information indicating an optical characteristic that influences sharpness of the image captured by the image capturing unit, for the packaging material that packs the verification target medicine; and a correction processing unit configured to execute correction processing of correcting the sharpness of the master image when the optical characteristic indicated by the characteristic information acquired by the characteristic information acquisition unit is an optical characteristic that decreases the sharpness of the image of the verification target medicine captured by the image capturing unit to be lower than the sharpness of the master image, in which, when the correction processing unit has executed the correction processing, the verification unit verifies the type of the verification target medicine using the master image in which the sharpness has been corrected.

Furthermore, it is preferable that the correction processing unit executes the correction processing of decreasing the sharpness of the master image in accordance with the optical characteristic indicated by the characteristic information acquired by the characteristic information acquisition unit.

Furthermore, it is preferable that the correction processing unit executes the correction processing of decreasing the sharpness of the master image in accordance with the optical characteristic indicated by the characteristic information acquired by the characteristic information acquisition unit, by using an image processing technique for blurring an image.

Furthermore, it is preferable to have a configuration further including a measurement unit configured to measure an optical characteristic of the packaging material, in which the measurement unit is provided inside the medicine verification device, and the characteristic information acquisition unit acquires the characteristic information indicating the optical characteristic measured by the measurement unit.

Furthermore, it is also allowable to have a configuration further including a measurement unit configured to measure an optical characteristic of the packaging material, the measurement unit being provided outside the medicine verification device, in which the characteristic information acquisition unit acquires the characteristic information indicating the optical characteristic measured by the measurement unit by an input of the characteristic information indicating the optical characteristic measured by the measurement unit, from the measurement unit to the medicine verification device.

Furthermore, it is preferable to have a configuration in which the packaging material is a bag-shaped packaging bag, further provided with a conveyance unit configured to convey a strip-shaped continuous packaging bag including continuously arranged packaging bags along a conveyance path, the image capturing unit captures an image for each of the packaging bags at an intermediate position of the conveyance path, and the measurement unit measures an optical characteristic for at least one packaging bag out of the continuous packaging bag at an intermediate position of the conveyance path.

Furthermore, it is preferable to further include a light irradiation unit configured to emit light to the packaging bag within an imaging range of the image capturing unit out of the continuous packaging bag when the image capturing unit captures the image.

Furthermore, it is preferable to have a configuration in which the continuous packaging bag includes an empty packaging bag containing no medicine, and the measurement unit measures an optical characteristic of the empty packaging bag.

Moreover, it is preferable that the characteristic information acquisition unit acquires characteristic information indicating at least one of the light transmissivity and the light scattering characteristic of the packaging material.

Furthermore, it is preferable that the characteristic information acquisition unit acquires the characteristic information indicating at least one of light transmittance of the packaging material, a haze value, and a Modulation Transfer Function curve indicating a correspondence relationship between a contrast and a spatial frequency.

Furthermore, it is preferable to have a configuration further including a prescription condition acquisition unit configured to acquire prescription condition information indicating prescription conditions set for prescribing a medicine, in which the verification unit verifies the type of the verification target medicine using the master image corresponding to the type of the medicine specified by the prescription condition information acquired by the prescription condition acquisition unit and the image of the verification target medicine captured by the image capturing unit.

Furthermore, it is preferable to have a configuration further including an optical characteristic determination unit configured to determine whether the optical characteristic indicated by the characteristic information acquired by the characteristic information acquisition unit is an optical characteristic of a case that the sharpness of the image of the verification target medicine captured by the image capturing unit is lower than the sharpness of the master image, in which the correction processing unit executes the correction processing when the optical characteristic determination unit determines that the optical characteristics indicated by the characteristic information acquired by the characteristic information acquisition unit is the optical characteristic that decreases the sharpness of the image of the verification target medicine captured by the image capturing unit to be lower than the sharpness of the master image.

Furthermore, it is preferable that the optical characteristic determination unit specifies a magnitude relationship between the optical characteristic indicated by the characteristic information acquired by the characteristic information acquisition unit and the optical characteristic of the packaging material packing the medicine appearing in the master image, and thereby determines whether the optical characteristic indicated by the characteristic information acquired by the characteristic information acquisition unit is the optical characteristic that decreases the sharpness of the image of the verification target medicine captured by the image capturing unit to be lower than the sharpness of the master image.

Furthermore, in order to achieve the above object, a medicine verification method of the present invention includes: a step of capturing, by an image capturing unit, an image of a verification target medicine packed in a packaging material having light transmissivity; a step of verifying a type of the verification target medicine using a master image registered in association with the type of medicine and an image of the verification target medicine captured by the image capturing unit; a step of acquiring characteristic information indicating an optical characteristic that influences sharpness of the image captured by the image capturing unit, for the packaging material that packs the verification target medicine; and a step of executing correction processing of correcting the sharpness of the master image when the optical characteristic indicated by the characteristic information thus acquired is an optical characteristic that decreases the sharpness of the image of the verification target medicine captured by the image capturing unit to be lower than the sharpness of the master image, in which, when the correction processing has been executed, the type of the verification target medicine is verified using the master image in which the sharpness has been corrected.

Furthermore, it is preferable that the above medicine verification method further includes a step of determining whether the optical characteristic indicated by the characteristic information thus acquired is an optical characteristic that decreases the sharpness of the image of the verification target medicine captured by the image capturing unit to be lower than the sharpness of the master image.

According to the medicine verification device and the medicine verification method of the present invention, in a case where the master image and the captured image of the verification target medicine have difference in the sharpness, the master image is corrected and the type of medicine is verified using the corrected master image. This makes it possible to eliminate the influence of the difference in the sharpness between the captured image of the verification target medicine and the master image on the verification result, leading to achievement of accurate verification of the type of medicine.

### BRIEF DESCRIPTION OF THE DRAWINGS

FIG. 1 is a diagram illustrating a flow of a medicine prescription operation.

FIG. 2 is a view illustrating a continuous packaging bag.

FIG. 3 is a schematic view illustrating an internal structure of a device main body included in a medicine verification device according to one embodiment of the present invention.

FIG. 4 is a view illustrating an imaging range of an image capturing unit and a part of a continuous packaging bag included in the range.

FIG. 5 is a schematic top view of a plurality of light emitting units of a light irradiation unit.

FIG. 6 is a block diagram illustrating a configuration of a processing device included in a medicine verification device according to one embodiment of the present invention.

FIG. 7 is a diagram illustrating a database in which master images are registered.

FIG. 8 is a diagram illustrating a master image management table.

FIG. 9 is a diagram illustrating a general verification flow.

FIG. 10 is a view illustrating a medicine extraction image.

FIG. 11 is a diagram illustrating a flow of a basic operation of a medicine verification device according to one embodiment of the present invention.

FIG. 12 is a schematic diagram illustrating a method of measuring optical characteristics of a packaging material using an integrating sphere.

FIG. 13 is a diagram illustrating a Modulation Transfer Function (MTF) curve.

### DETAILED DESCRIPTION OF THE INVENTION

Hereinafter, a medicine verification device and a medicine verification method of the present invention will be described in detail.

Although the description of the components described below might be made based on typical embodiments of the present invention, the present invention is not limited to such embodiments. That is, the following embodiments are an example provided to facilitate understanding of the medicine verification device and the medicine verification method of the present invention and would not limit the present invention. Accordingly, various improvements or alterations may be made without departing from the scope and spirit of the present invention.

In addition, in the present description, a “medicine” represents a solid medicine, and specifically corresponds to a tablet or a capsule.

<<Medicine Prescription Operation>>

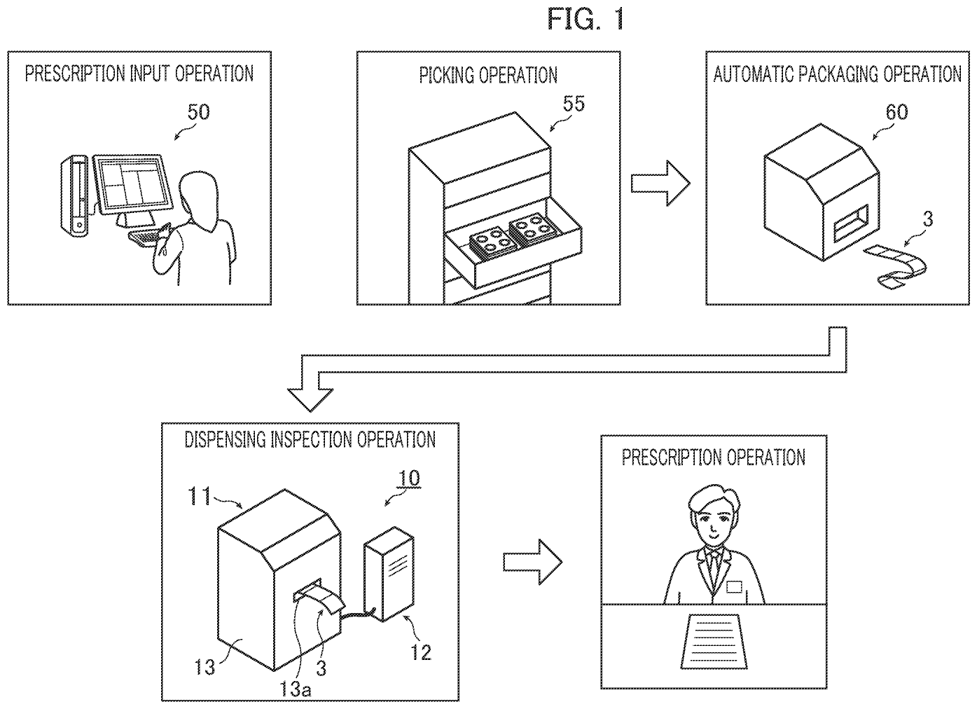

Before describing the medicine verification device (hereinafter, a medicine verification device 10) according to one embodiment of the present invention, a medicine prescription operation performed using the medicine verification device 10 will be outlined first. The medicine prescription operation includes operations sequentially performed in the order of a prescription input operation, a picking operation, an automatic packaging operation, a dispensing inspection operation, and a prescription operation, as illustrated in FIG. 1. FIG. 1 is a diagram illustrating a flow of the medicine prescription operation.

In the prescription input operation, a pharmacist inputs prescription conditions described in a prescription to a computer (hereinafter, a prescription condition input device 50). Here, a prescription condition is a condition set for prescribing a medicine to a patient. Examples of input prescription conditions include the name and age of the patient, the type of medicine to be prescribed, the prescription quantity for each of types. The following description assumes that medicines are taken by a plurality of doses and that the prescription quantity for one dose is the same. However, the present invention is not limited to this, and a medicine for only one dose may be prescribed. Furthermore, the type and the prescription quantity of the medicine for one dose may be different each of times.

In the picking operation, on the basis of prescription conditions, a pharmacist picks medicines of the types corresponding to the prescription conditions from a medicine shelf 55 by the quantities according to the prescription conditions. Note that the picking operation is not limited to the case where the pharmacist performs the operation manually, but may be performed automatically by a known automatic picking device on the basis of the prescription conditions input to the prescription condition input device 50.

Furthermore, each of the medicines picked in the present embodiment includes identification information formed on a medicine surface. The “identification information” includes characters, numerals, symbols, or the like for identifying the type of medicine (medicine type) and is formed by engraving or printing. In the present embodiment, it is assumed that identification information is formed on the surface of the medicine by engraving (recess processing). However, the present invention is not limited to the above-described embodiment, and medicines to be picked may include medicines for which identification information is not formed or may include medicines for which identification information is formed by printing.

In the automatic packaging operation, the pharmacist sets the medicines picked in the picking operation onto a tray of a packaging machine 60 illustrated in FIG. 1, and then, the packaging machine 60 automatically packages the medicines in the tray. At this time, the picked medicines are set on a tray for one dose, and the medicines for one dose are packaged in each of the plurality of packaging bags 1. The packaging bag 1 is a bag-shaped sachet and is formed of a packaging material having light transmissivity. Examples of the material of the packaging bag 1 include a laminated film of cellophane and polyethylene, and a polyethylene film.

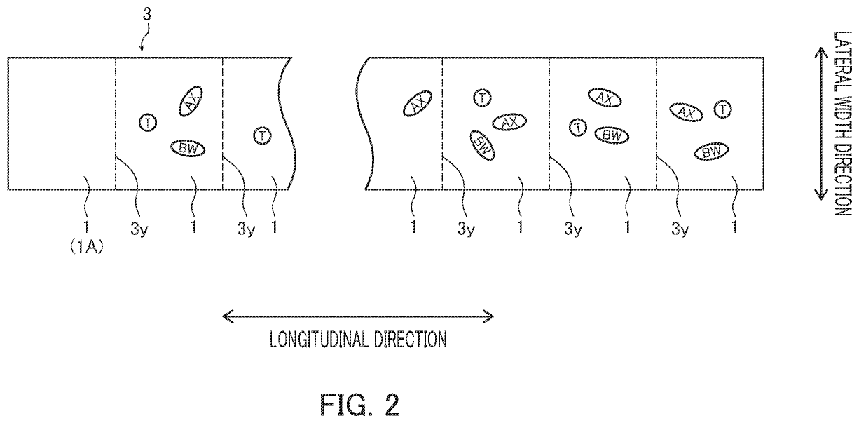

At a point of completion of the automatic packaging operation, the plurality of packaging bags 1 each containing the medicines are continuously arranged to form a continuous packaging bag 3 having a strip-like shape as illustrated in FIG. 2. At the time of taking the medicine, one packaging bag 1 is separated from the continuous packaging bag 3 and the medicine packed in the separated packaging bag 1 is taken. FIG. 2 is a view illustrating the continuous packaging bag 3.

Note that the plurality of packaging bags 1 are not limited to the form of being continuous as the continuous packaging bag 3 at a point of completion of the automatic packaging operation and may be separated from each other.

At a point of completion of the automatic packaging operation, the packaging bag 1 located at one end of the continuous packaging bag 3 is an empty packaging bag 1 containing no medicine (hereinafter, referred to as an empty bag 1A) as illustrated in FIG. 2. The empty bag 1A is similar to the packaging bag 1 packing the medicine, except for not containing the medicine inside. The empty bag 1A may be provided at a location other than the end of the continuous packaging bag 3. Moreover, there is no need to include the empty bag 1A in the continuous packaging bag 3.

In the dispensing inspection operation, inspection of whether the prescribed medicine is correct is performed using the medicine verification device 10 illustrated in FIG. 1. Specifically, inspection of whether the type and the number (more precisely, the number of each of types) of the medicines contained in each of the packaging bags 1 in the continuous packaging bag 3 is as specified in the prescription is performed.

The prescription operation performs prescription of prepackaged medicines determined to be correct (as instructed by the prescription) in the dispensing inspection operation, for the patient (prescription destination). At this time, the pharmacist removes the empty bag 1A located at one end of the continuous packaging bag 3 and hands the remaining continuous packaging bag 3 to the patient.

<<Configuration of Medicine Verification Device>>

Next, a configuration of the medicine verification device 10 will be described.

The medicine verification device 10 is used for dispensing inspection and verifies the type and the number (more precisely, the number of each type) of the medicine packed in the packaging bag 1 in the automatic packaging operation. Here, the medicine packed (packaged) in one packaging bag 1 corresponds to the “verification target medicine” of the present invention.

As illustrated in FIG. 1, the medicine verification device 10 includes: a device main body 11 having a function of capturing an image of a medicine as a verification target (specifically, a medicine packed in each of the packaging bags 1); and a processing device 12 having a function of performing dispensing inspection based on the image captured by the device main body 11.

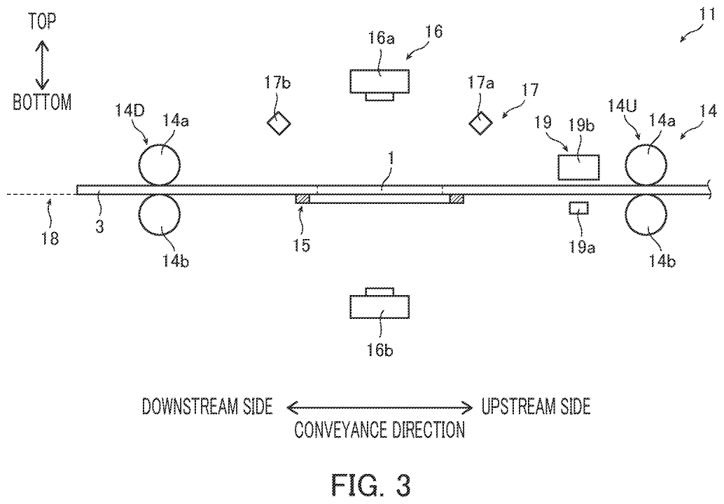

The device main body 11 includes a housing 13 illustrated in FIG. 1, and includes, within the housing 13, a conveyance unit 14, an arrangement unit 15, an image capturing unit 16, and a light irradiation unit 17 illustrated in FIG. 3. FIG. 3 is a schematic view illustrating an internal structure of the device main body 11. In addition, the housing 13 of the device main body 11 includes: an introduction part 13a for introducing the continuous packaging bag 3 to the inside of the device main body 11; and a discharge part (not illustrated) for discharging the continuous packaging bag 3 introduced inside the device main body 11 to the outside of the device main body 11.

The conveyance unit 14 has a conveyance path 18 formed inside the device main body 11 and conveys the continuous packaging bag 3 along the conveyance path 18. The continuous packaging bag 3 introduced into the inside of the device main body 11 from the introduction part 13a moves toward the downstream side of the conveyance path 18 by the conveyance operation of the conveyance unit 14 and eventually passes through the discharge part to be discharged to the outside of the device main body 11. Here, the “downstream side” means a side closer to the discharge part in the conveyance direction, and an “upstream side” means a side opposite to the downstream side, that is, a side closer to the introduction part 13a in the conveyance direction.

In the present embodiment, the conveyance path 18 is a horizontal path, and the conveyance unit 14 performs conveyance in a state where a longitudinal direction of the continuous packaging bag 3 runs along the conveyance path 18 (that is, the conveyance direction) and where a thickness direction of the continuous packaging bag 3 runs along the up-down direction (vertical direction).

As illustrated in FIG. 3, the conveyance unit 14 includes an upstream drive unit 14U and a downstream drive unit 14D. The upstream drive unit 14U is arranged on the upstream side of the arrangement unit 15, while the downstream drive unit 14D is arranged on the downstream side of the arrangement unit 15. Each of the upstream drive unit 14U and the downstream drive unit 14D includes a pair of upper and lower nip rollers 14a and 14b, and a motor (not illustrated) that rotationally drives one of the pair of upper and lower nip rollers 14a and 14b. The pair of upper and lower nip rollers 14a and 14b is arranged with a gap enough to allow the continuous packaging bag 3 to pass through, and the rollers rotate in a state of nipping the continuous packaging bag 3 between the rollers. With this configuration, the continuous packaging bag 3 is conveyed in a state where a slight tension is applied.

In the present embodiment, the motor is configured to rotate intermittently. Therefore, the conveyance unit 14 performs the conveyance operation intermittently. In one conveyance operation, the continuous packaging bag 3 moves by a predetermined amount in the conveyance direction. The movement amount (conveyance amount) of the continuous packaging bag 3 in one conveyance operation is set by the control unit 21 of the processing device 12 described below.

Note that the conveyance unit 14 of the present embodiment can perform conveyance to either the upstream side or the downstream side in the conveyance direction by switching the rotational direction of the motor. The conveyance direction is set by the control unit 21 of the processing device 12.

Furthermore, while the present embodiment is an example that applies the conveyance mechanism using the rotation drive of the roller (that is, a roller conveyor), it is also allowable to use other conveyance mechanisms as long as the mechanism can convey the continuous packaging bag 3 properly. For example, it is allowable to apply a belt conveyor that conveys the continuous packaging bag 3 by rotating an endless belt while the continuous packaging bag 3 is mounted on the upper surface of the belt.

The arrangement unit 15 is a portion where the verification target medicine is disposed in a state of being packed in the packaging bag 1 and is provided at an intermediate position of the conveyance path 18 as illustrated in FIG. 3. The arrangement unit 15 is a rectangular frame-shaped base and has a size on which one packaging bag 1 can be mounted. In addition, the packaging bags 1 disposed on the arrangement unit 15 in the continuous packaging bag 3 are sequentially switched together with the conveyance of the continuous packaging bag 3 by the conveyance unit 14.

Note that, in a state where the packaging bag 1 is disposed on the arrangement unit 15, the entire region of the upper surface of the packaging bag 1 (the surface facing the upper side of the device main body 11, the similar applies hereinafter) is exposed, while regions other than the edge of the lower surface of the packaging bag 1 (the surface facing the upper side of the device main body 11, the similar applies hereinafter) are exposed. The edge of the packaging bag 1 is a sealed portion formed by stacking and pressing two film sheets constituting the packaging bag 1 together.

The image capturing unit 16 captures, for each of the packaging bags 1, an image of the medicine packed in each of the packaging bags 1 in the continuous packaging bag 3, that is, an image of the verification target medicine, at an intermediate position of the conveyance path 18. More specifically, the image capturing unit 16 captures an image of the packaging bag 1 arranged at the arrangement unit 15 and an image of the medicine inside the packaging bag 1 every time the packaging bag 1 disposed on the arrangement unit 15 is switched.

As illustrated in FIG. 3, the image capturing unit 16 includes two cameras, upper and lower, as a plurality of cameras. The camera on the upper side (hereinafter, referred to as a first camera 16a) is disposed immediately above the arrangement unit 15 and captures an image of the medicine packed in the packaging bag 1 disposed on the arrangement unit 15 from above. The lower camera (hereinafter, referred to as a second camera 16b) is disposed immediately below the arrangement unit 15 and captures an image of the medicine packed in the packaging bag 1 disposed on the arrangement unit 15 from below. Here, the “medicine image” or “image of a medicine” means an image of the medicines captured through the packaging bag 1 in the present embodiment.

In the present embodiment, the conveyance operation by the conveyance unit 14 is to be performed intermittently, and the image capturing unit 16 captures an image of the packaging bag 1 arranged on the arrangement unit 15 and an image of a medicine packed in the packaging bag 1 between the conveyance operations. In addition, every time the packaging bag 1 disposed on the arrangement unit 15 is switched by the conveyance operation, the image capturing unit 16 captures the image of the packaging bag 1 disposed on the arrangement unit 15 and the image of the medicine packed in the packaging bag 1.

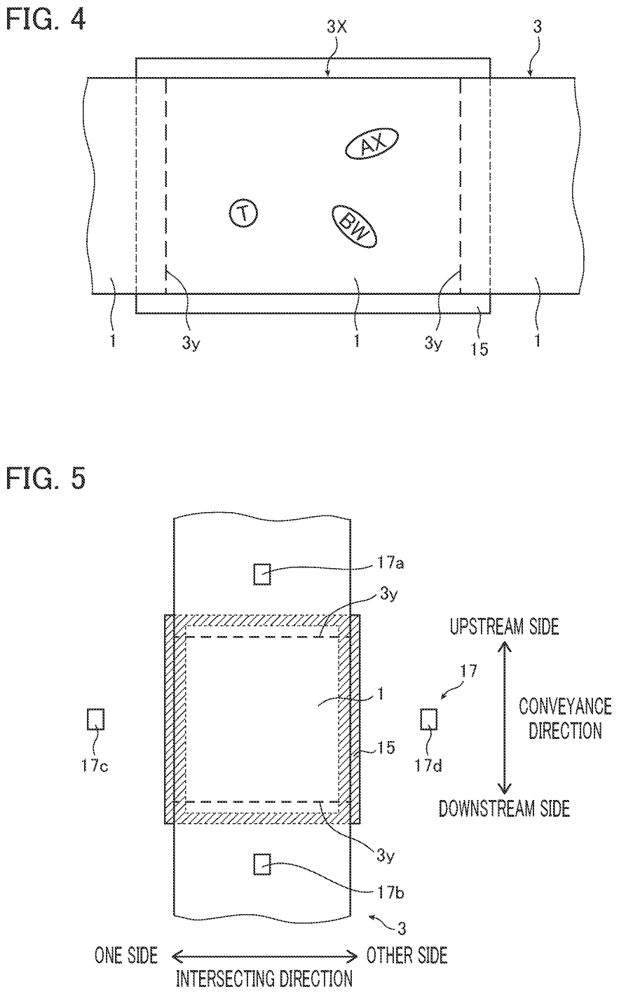

In the present embodiment, the imaging range of the first camera 16a is set to a rectangular region as illustrated in FIG. 4 (a rectangular region illustrated by a broken line in FIG. 4), which is a range capable of imaging an entire surface of the upper surface of the packaging bag 1 disposed on the arrangement unit 15 and a part (more precisely, an end portion) of the upper surface of the packaging bag 1 located on both sides of the packing bag 1 disposed on the arrangement unit 15. In other words, the portion of the continuous packaging bag 3 that is within the imaging range of the first camera 16a corresponds to an imaging target portion 3x, and at least the cutout line 3y between the packaging bags 3 is located at this portion as illustrated in FIG. 4. Here, the cutout line 3y is a boundary recess formed at a boundary position between the packaging bags 3 in the continuous packaging bag 3, and more specifically, is constituted by a dashed linear groove being formed from one end to the other end of the continuous packaging bag 3 in the lateral width direction of the continuous packaging bag 3.

FIG. 4 is a view illustrating an imaging range of the image capturing unit 16 and a part of the continuous packaging bag 3 included in the range.

Similarly, the imaging range of the lower camera 16b is set to a rectangular region, which is a range capable of imaging a region on the lower surface of the packaging bag 1 disposed on the arrangement unit 15 that is inside the arrangement unit 15 and is exposed, and a portion (more precisely, an end portion) of the lower surface of the packaging bag 1 located on both sides of the packing bag 1 disposed on the arrangement unit 15. In other words, the portion of the continuous packaging bag 3 that is within the imaging range of the second camera 16b corresponds to the imaging target portion 3x, and this portion includes at least the cutout line 3y between the packaging bags 1.

The image capturing unit 16 may be any type as long as it has a function of acquiring image data of a subject. Examples of this include a Charge-Coupled Device (CCD) image sensor and a Complementary Metal Oxide Semiconductor (CMOS) image sensor, although the present invention is not limited to these.

Furthermore, the image capturing unit 16 in the present embodiment is implemented by two cameras, but the number of cameras is not particularly limited and may be one, or three or more.

Furthermore, in the present embodiment, the camera is installed at a position vertically sandwiching the arrangement unit 15. However, the installation position of the camera can be set to any position as long as the packaging bag 1 disposed on the arrangement unit 15 and the medicine packed in the packaging bag 1 can be imaged satisfactorily.

The light irradiation unit 17 is configured to perform light emission to the packaging bag 1 disposed on the arrangement unit 15 and the medicine packed in the packaging bag 1 (that is, the verification target medicine) when the image capturing unit 16 captures an image. More specifically, when the image capturing unit 16 captures an image, the light irradiation unit 17 emits light toward the packaging bag 1 within an imaging range of the image capturing unit 16 of the continuous packaging bag 3 (more precisely, the surface of the imaging target portion 3x).

As illustrated in FIG. 5, the light irradiation unit 17 has a plurality of light emitting units, specifically four light emitting units 17a, 17b, 17c, and 17d in the present embodiment. FIG. 5 is a schematic top view of a plurality of light emitting units of the light irradiation unit 17. The four light emitting units 17a, 17b, 17c, and 17d are light sources used when the light irradiation unit 17 performs light emission, and individually arranged on four sides of the arrangement unit 15 as illustrated in FIG. 5. The light irradiation unit 17 performs light emission in different directions using the four light emitting units 17a, 17b, 17c, and 17d (a plurality of light emitting units).

More specifically, the two light emitting units 17a and 17b are arranged at positions opposite to each other as viewed from the arrangement unit 15 in the conveyance direction, and the units emit light in mutually opposite directions. That is, one light emitting unit 17a (hereinafter, referred to as a first light emitting unit 17a) emits light from the upstream side in the conveyance direction to the arrangement unit 15 located downstream in the conveyance direction. The other light emitting unit 17b (hereinafter, referred to as a second light emitting unit 17b) emits light from the downstream side in the conveyance direction to the arrangement unit 15 located upstream in the conveyance direction.

The remaining two light emitting units 17c and 17d out of the four light emitting units 17a, 17b, 17c, and 17d are arranged at positions opposite to each other as viewed from the arrangement unit 15 in a direction (hereinafter, an intersecting direction) intersecting the conveyance direction, and the units emit light in mutually opposite directions. That is, one light emitting unit 17c (hereinafter, referred to as a third light emitting unit 17c) emits light from one side in the intersecting direction toward the arrangement unit 15 on the other side. The other light emitting unit 17d (hereinafter, referred to as a fourth light emitting unit 17d) emits light to the arrangement unit 15 on one side, from the other side, in the intersecting direction. Here, “one side in the intersecting direction” means, for example, a side near one end of the arrangement unit 15 in the intersecting direction, while “the other side in the intersecting direction” means a side close to the other end of the arrangement unit 15 in the intersecting direction.

The light irradiation unit 17 uses a part or all of the four light emitting units 17a, 17b, 17c, and 17d to perform light emission to the packaging bag 1 disposed on the arrangement unit 15 and the medicine packed in the packaging bag 1. At this time, as observed from FIGS. 3 and 5, the light irradiation unit 17 performs light emission diagonally to the packaging bag 1 disposed on the arrangement unit 15 and the medicine (that is, the imaging target portion 3x) packed in the packaging bag 1. This direction is advantageous because applying light diagonally to the surface of the medicine can emphasize the contour of the identification information formed on the surface of the medicine (in particular, an edge portion of the contour at the object of light emission).

In addition, in the present embodiment, it is possible to switch the light emitting units 17a, 17b, 17c, and 17d used by the light irradiation unit 17 when the image capturing unit 16 captures an image. Specifically, the light irradiation unit 17 performs light emission using one of the four light emitting units 17a, 17b, 17c, and 17d. While the light irradiation unit 17 performs light emission from one light emitting unit, the image capturing unit 16 captures an image of the medicine packed in the packaging bag 1 disposed on the arrangement unit 15, by one imaging. Thereafter, the light irradiation unit 17 switches one light emitting unit used immediately before to another light emitting unit among the light emitting units 17a, 17b, 17c, and 17d and then, performs light emission using the light emitting unit 17a, 17b, 17c, or 17d after the switching. In the meantime, the image capturing unit 16 captures an image of the medicine packed in the packaging bag 1 disposed on the arrangement unit 15 again.

Thereafter, the light irradiation unit 17 sequentially switches the light emitting units 17a, 17b, 17c, and 17d using the similar procedure, and the image capturing unit 16 captures an image of the medicine packed in the packaging bag 1 disposed on the arrangement unit 15 every time the light irradiation unit 17 switches the light emitting units 17a, 17b, 17c, and 17d. This results in acquisition of captured images for each of the light irradiation directions (that is, four images having mutually different reflection states of light at each of portions of the medicine surface) for the medicine packed in one packaging bag 1 disposed on the arrangement unit 15. However, the present invention is not limited to this, and it is allowable to use a configuration in which two to four of the four light emitting units 17a, 17b, 17c, and 17d are simultaneously turned on, and the light irradiation unit 17 performs light emission toward the medicine packed in one packaging bag 1 disposed on the arrangement unit 15 simultaneously from two to four directions.

The light emitting units 17a, 17b, 17c, and 17d used by the light irradiation unit 17 for light emission may be implemented with known light sources, including any of a point light source, a line light source, or a surface light source. Specifically, examples of applicable light sources include: electroluminescence types such as Light Emitting Diode (LED), semiconductor laser (Laser Diode (LD)), and organic Electroluminescence (EL), radiant heat types such as halogen bulbs and incandescent bulbs, discharge emission types such as a mercury lamp and a fluorescent lamp, and a combination of these light sources with a light guide member such as a light guide plate or an optical fiber.

Furthermore, the present embodiment uses the light irradiation unit 17 having four light emitting units 17a, 17b, 17c, and 17d. However, the number of light emitting units (light sources) is not particularly limited, and it is sufficient as long as two or more units are provided.

Moreover, instead of arranging the four light emitting units 17a, 17b, 17c, and 17d around the arrangement unit 15, it is allowable to dispose one annular light emitting unit. With such a configuration, it is possible to irradiate the arrangement unit 15 with light from all directions of 360 degrees.

In addition to the parts described above (specifically, the conveyance unit 14, the arrangement unit 15, the image capturing unit 16, and the light irradiation unit 17), the device main body 11 also includes a measurement unit 19 illustrated in FIG. 3. The measurement unit 19 measures an optical characteristic of the packaging bag 1. Here, the “optical characteristic of the packaging bag 1” refers to a characteristic of the packaging bag 1 that influences the sharpness (in other words, the degree of blurring) of the image of the medicine packaged in the packaging bag 1 captured by the image capturing unit 16, and more specifically refers to the light transmissivity and light scattering characteristics of the packaging bag 1. The measurement unit 19 according to the present embodiment measures the light transmittance of the packaging bag 1 as the characteristic (light transmissivity) of the packaging bag 1. However, the present invention is not limited to this, and a haze value (haze) may be measured as an optical characteristic of the packaging bag 1.

In the present embodiment, the measurement unit 19 is provided inside the device main body 11 of the medicine verification device 10. The measurement unit 19 has a configuration similar to a known light transmittance meter and includes a light source 19a and a light receiver 19b as illustrated in FIG. 3. The light source 19a and the light receiver 19b are arranged upstream of the arrangement unit 15 in the conveyance direction. A part of the conveyance path 18 is interposed between the light source 19a and the light receiver 19b in the up-down direction. Therefore, each of portions in the continuous packaging bag 3 (that is, each of the packaging bags 1) passes between the light source 19a and the light receiver 19b when being conveyed by the conveyance unit 14. The measurement unit 19 emits, from the light source 19a, light of a specific wavelength toward the packaging bag 1 disposed between the light source 19a and the light receiver 19b and receives the light transmitted through the packaging bag 1 by the light receiver 19b. Subsequently, the measurement unit 19 obtains the light transmittance based on the intensity of the light emitted from the light source 19a and the intensity of the light received by the light receiver 19b. The wavelength of the light emitted from the light source 19a can be set arbitrarily.

Furthermore, in the present embodiment, the measurement unit 19 measures an optical characteristic of the packaging bag 1 for at least one packaging bag 1 of the continuous packaging bag 3 at an intermediate position of the conveyance path 18. More specifically, in the present embodiment, the measurement unit 19 measures the light transmittance of the empty packaging bag 1 containing no medicine (that is, the empty bag 1A) of the continuous packaging bag 3. This makes it possible to measure the light transmittance of the packaging bag 1 with higher accuracy. However, the present invention is not limited to this, and the light transmittance may be measured for the packaging bag 1 containing a medicine.

Furthermore, in the present embodiment, the measurement unit 19 measures the light transmittance of the empty bag 1A present in the continuous packaging bag 3 every time a new continuous packaging bag 3 is introduced into the device main body 11.

The processing device 12 executes a series of information processing in the implementation of the dispensing inspection. In the present embodiment, the processing device 12 is constituted using a personal computer (PC) external to the device main body 11. However, the present invention is not limited to this, and the processing device 12 may be constituted using a computer built in the device main body 11.

Furthermore, the processing device 12 is communicably connected to the device main body 11, the prescription condition input device 50, and a server computer 70 described below. The medicine verification device 10 constitutes a medicine verification system together with the server computer 70 connected to the processing device 12 via a network. The connection method between the processing device 12 and each of devices may be a wired connection method or a wireless connection method.

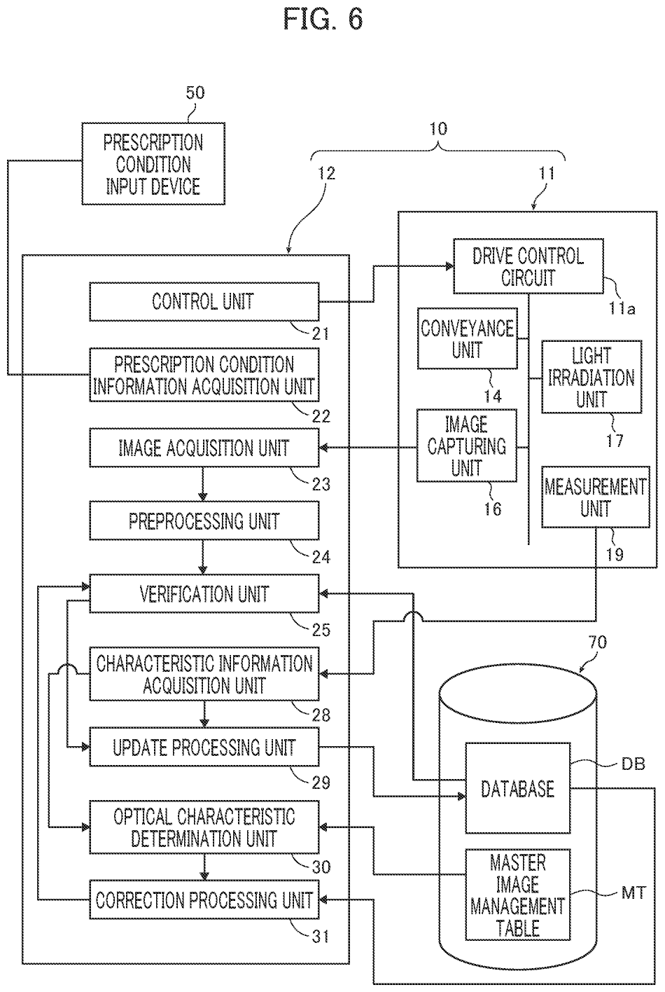

Furthermore, as illustrated in FIG. 6, the processing device 12 includes a control unit 21, a prescription condition information acquisition unit 22, an image acquisition unit 23, a preprocessing unit 24, a verification unit 25, a characteristic information acquisition unit 28, an update processing unit 29, an optical characteristic determination unit 30, and a correction processing unit 31. FIG. 6 is a block diagram illustrating a configuration of the processing device 12. These individual units are implemented by cooperation of hardware devices such as a Central Processing Unit (CPU) and a memory (not illustrated) included in the processing device 12, and an information processing program stored in the processing device 12. The information processing program may be read and obtained from a recording medium such as a Compact Disc Read Only Memory (CD-ROM) storing the program, or may be downloaded and obtained from a predetermined site via a network.

In the present embodiment, individual functional units of the processing device 12 (specifically, the control unit 21, the prescription condition information acquisition unit 22, the image acquisition unit 23, the preprocessing unit 24, the verification unit 25, the characteristic information acquisition unit 28, the update processing unit 29, the optical characteristic determination unit 30, and the correction processing unit 31) are constituted by one personal computer. However, the present invention is not limited to this, and it is allowable to use a configuration in which a part of the above functional units is constituted by one personal computer while the remaining functional units are constituted by another personal computer.

The control unit 21 is electrically connected to each of units of the device main body 11 (specifically, the conveyance unit 14, the image capturing unit 16, the light irradiation unit 17, and the measurement unit 19) via a drive control circuit 11a mounted on the device main body 11 and controls each of the units of the device.

More specifically, the control unit 21 performs control related to the conveyance operation of the conveyance unit 14, such as controlling a conveyance amount, a conveyance direction, a conveyance operation timing, or the like in one conveyance operation. In addition, the control unit 21 performs control related to the imaging operation of the image capturing unit 16, such as controlling a camera to be used among the two cameras 16a and 16b of the image capturing unit 16, and a timing of imaging. In addition, the control unit 21 performs control related to the light emitting operation of the light irradiation unit 17, such as controlling the light emitting unit to be used among the four light emitting units 17a, 17b, 17c, and 17d included in the light irradiation unit 17, and the timing of light emission.

Furthermore, the control unit 21 controls the measurement timing (measurement start point) regarding the light transmittance measurement performed by the measurement unit 19. Specifically, at a time when the continuous packaging bag 3 is introduced into the device main body 11 and the leading end of the continuous packaging bag 3 (the end located on the most downstream side in the conveyance direction) passes through a position immediately before a space between the light source 19a and the light receiver 19b of the measurement unit 19 in the conveyance path 18, the control unit 21 turns on the light source 19a and causes the measurement unit 19 to start light transmittance measurement.

The prescription condition information acquisition unit 22 is communicably connected to the prescription condition input device 50 and acquires prescription condition information by communicating with the prescription condition input device 50. Here, the prescription condition information is information indicating the prescription condition, which specifically is electronic data indicating the prescription condition input to the prescription condition input device 50 by the pharmacist.

In the present embodiment, when the input of the prescription condition is completed in the prescription condition input device 50, the prescription condition information is automatically transmitted from the prescription condition input device 50 to the prescription condition information acquisition unit 22, and then the prescription condition information acquisition unit 22 receives the above prescription condition information. However, the present invention is not limited to this. It is also allowable to use a configuration in which an information transmission request is transmitted from the prescription condition information acquisition unit 22, and the prescription condition input device 50 transmits the prescription condition information at a point of reception of the request by the prescription condition input device 50. More specifically, character string information or the two-dimensional barcode information for specifying the prescription condition is printed on a tip portion of the continuous packaging bag 3 (a portion of the continuous packaging bag 3 first introduced into the device main body 11). The prescription condition information acquisition unit 22 reads the above-described printed information when the continuous packaging bag 3 is introduced into the device main body 11. Thereafter, based on the read printed information, the prescription condition information acquisition unit 22 requests a prescription condition information indicating prescription conditions related to the medicine packaged in each of the packaging bags 1 of the continuous packaging bag 3 introduced into the device main body 11, against the prescription condition input device 50. After receiving this request, the prescription condition input device 50 analyzes the request, specifies prescription condition information related to the request, and transmits the specified prescription condition information to the processing device 12.

The image acquisition unit 23 is connected to the image capturing unit 16 (more precisely, the first camera 16a and the second camera 16b) and acquires, via a network, an image captured by the image capturing unit 16. Here, the image acquired by the image acquisition unit 23 corresponds to image data in specifically, Joint Photographic Experts Group (JPEG) format, Graphics Interchange Format (GIF) format, Portable Network Graphics (PNG) format, Tagged Image File Format (TIFF), Bitmap Image (BMP) format, or the like.

Note that the image acquisition unit 23 acquires an image from the image capturing unit 16 each time the image capturing unit 16 captures an image. More specifically, in the present embodiment, as described above, images are captured a plurality of times (specifically, eight times) separately for each of imaging conditions for one packaging bag 1 in which medicines are packaged. Accordingly, the image acquisition unit 23 acquires images for various imaging conditions (that is, eight images) for each of the packaging bags 1 and the medicines packed in the packaging bags 1. Furthermore, when the packaging bag 1 disposed on the arrangement unit 15 is switched, the image capturing unit 16 newly captures an image for each of imaging conditions. Accordingly, the image acquisition unit 23 acquires the newly captured image for each of the imaging conditions.

The preprocessing unit 24 performs preprocessing on the image acquired by the image acquisition unit 23 from the image capturing unit 16 (that is, the image data of the verification target medicine). The preprocessing is a process for emphasizing the identification information formed on the surface of the medicine appearing in the image acquired by the image acquisition unit 23.

More specifically, in the present embodiment, as described above, an image is captured a plurality of times (specifically, four times) in various light irradiation directions for the medicine packed in one packaging bag 1. Here, each of the images for each of the light irradiation directions has unevenness in light illuminance occurring on the surface of the medicine appearing in the image. Such uneven illuminance of light has an influence in detecting and specifying identification information formed on the surface of the medicine. Furthermore, the illuminance unevenness of light varies depending on the light irradiation direction. Therefore, the preprocessing unit 24 performs preprocessing. For an image captured for each of light irradiation directions, the preprocessing uses an edge extraction filter in a direction corresponding to the irradiation direction, which is an edge extraction filter of a size corresponding to the number of pixels of an edge (marked groove) of the identification information appearing in each of the images, thereby generating an edge image for each of irradiation directions, and thereafter, generates a combined image in which a plurality of edge images are combined. The edge extraction filter can include at least one of a Sobel filter, a Laplacian filter, and a Canny filter, and can be appropriately selected according to a verification method to be described below.

The image that has undergone the above preprocessing is an image from which the illuminance unevenness of light that varies according to the light irradiation direction is eliminated as much as possible and in which the identification information formed on the surface of the medicine appearing in the image is emphasized.

Specifically, it is possible to reduce the information other than the engraving, such as the pattern and the scratches smaller than the groove of engraving indicating the identification information on the surface of the medicine, leading to extraction of the engraving.

The verification unit 25 verifies the number and type of medicines (that is, verification target medicine) packed in the packaging bag 1 disposed on the arrangement unit 15. More specifically, the verification unit 25 uses a master image corresponding to the type of medicine specified from the prescription condition information and an image of the verification target medicine captured by the image capturing unit 16 (more precisely, an image preprocessed by the preprocessing unit 24). Subsequently, the verification unit 25 uses these images to verify the type of verification target medicine and the number of medicines for each of types.

The specific procedure of verification performed by the verification unit 25 will be described below in detail.

Here, the master image will be described. The master image is an image of a medicine registered corresponding to the type of medicine and is an image registered in advance for the type of medicine specified from the prescription condition information. Furthermore, in the present embodiment, the master image is obtained from an image of a medicine captured in a state of being packed in the packaging bag 1.

Furthermore, regarding the medicine that has been verified by the verification unit 25 in the past, the captured image used in the past verification (more precisely, a medicine extraction image X described below) will be used as a master image in the subsequent verifications. More specifically, in a case where the verification unit 25 has verified that the type of the verification target medicine matches the type of the medicine appearing in the master image, the captured image of the verification target medicine is registered as a new master image and will be used as a master image in the subsequent verifications.

Note that there may be a case where a master image has not been registered for a new medicine or the like. For a medicine of a type for which a master image is not registered, a captured image obtained when the image capturing unit 16 first captures the medicine (more precisely, the medicine extraction image X) is to be registered as a master image. The following description will assume a case where master images are registered in advance for all the verification target medicines.

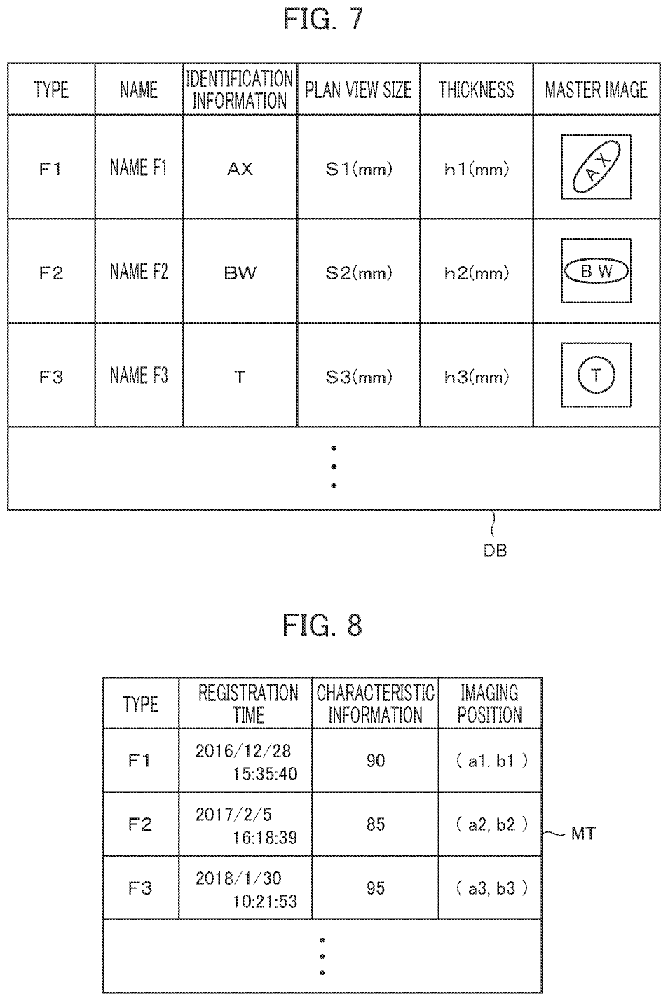

Furthermore, in the present embodiment, the master image is registered in the database DB, and the database DB is stored in the external server computer 70. The database DB will be described. As illustrated in FIG. 7, the database DB is a database in which a master image of each of medicines and a type of the medicine are registered in association with each other. FIG. 7 is a diagram illustrating a database DB in which master images are registered.

In addition to the master image, the database DB includes a medicine name, identification information formed on the surface of the medicine, a plan view size and a thickness as medicine size individually registered in association with the type of the medicine. The information registered in the database DB is not limited to the above information, and information other than the above information may be registered.

The verification unit 25 communicates with the server computer 70 to access the database DB, and reads from the database DB a master image corresponding to the type of medicine specified by the prescription condition information acquired by the prescription condition information acquisition unit 22.

In the present embodiment, the server computer 70 stores the master image (more precisely, the database DB in which the master image is registered). However, the present invention is not limited to this, and the master image may be stored in a storage medium in the processing device 12.

In addition, in the present embodiment, a master image management table MT is recorded in the server computer 70 in addition to the above database DB. The master image management table MT records information regarding the latest master image registered in the database DB, and records the type of medicine, registration time, characteristic information, and imaging position for each of the master images, as illustrated in FIG. 8. FIG. 8 is a diagram illustrating the master image management table MT.

Regarding the information recorded in the master image management table MT, the type of medicine is information indicating the type of medicine appearing in the master image. The registration time is information indicating the registration time or the latest update time for the master image. Characteristic information is the characteristic information of the packaging bag 1 appearing in the master image, more specifically refers to information indicating the light transmittance measured by the measurement unit 19 of the medicine verification device 10 in the past regarding the packaging bag 1 packing the medicine appearing in the master image.

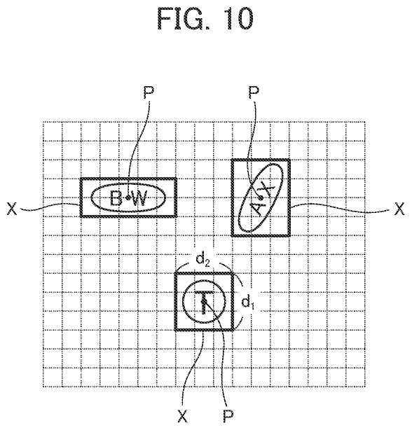

The imaging position is a position of the medicine with respect to the image capturing unit 16 at a time when the medicine image registered as the master image is captured by the image capturing unit 16. More specifically, the imaging position is a coordinate position when the reference position is the origin and the conveyance direction and the intersecting direction are the coordinate axis directions, and this corresponds to the center position of the medicine image registered as the master image (specifically, corresponds to the coordinates of point P illustrated in FIG. 10). In the present embodiment, the reference position being the origin is set at the center position of the imaging range (more precisely, the angle of view of each of the first camera 16a and the second camera 16b) of the image capturing unit 16. However, the present invention is not limited to this, and the reference position may be set to any position.

Note that the information recorded in the master image management table MT is not limited to the above-described information and may include information other than the above-described information. Furthermore, while the master image management table MT is stored in the server computer 70 in the present embodiment, the present invention is not limited to this. The master image management table MT may be stored in the storage medium of the processing device 12.

The characteristic information acquisition unit 28 acquires characteristic information. In the present embodiment, the characteristic information refers to information indicating the optical characteristic of the packaging bag 1 described above, specifically, information indicating at least one of the light transmissivity or the light scattering characteristic of the packaging bag 1, and more specifically refers to the information indicating the light transmittance of the packaging bag 1.

More specifically, in the present embodiment, the characteristic information acquisition unit 28 acquires a measurement result of the measurement unit 19 as the characteristic information. That is, when the measurement unit 19 has measured the light transmittance of the packaging bag 1 for the empty bag 1A in the continuous packaging bag 3, the characteristic information acquisition unit 28 acquires, from the measurement unit 19, the measurement result (that is, the light transmittance of the packaging bag 1) obtained by the measurement unit 19, through the transmission path (not illustrated) included in the measurement unit 19.

As described above, the characteristic information acquisition unit 28 acquires information indicating the light transmittance of the empty bag 1A in the continuous packaging bag 3, in which the light transmittance is the same between the empty bag 1A and other packaging bag 1 in the continuous packaging bag 3. Therefore, by acquiring the information indicating the light transmittance of the empty bag 1A in the continuous packaging bag 3, the characteristic information acquisition unit 28 acquires characteristic information (information indicating the light transmittance) for all the packaging bags 1 of the continuous packaging bag 3 introduced into the device main body 11. However, the present invention is not limited to this, and the measurement unit 19 may measure the light transmittance of each of the packaging bags 1 in the continuous packaging bag 3 one by one, and the characteristic information acquisition unit 28 may acquire the information indicating the light transmittance of the packaging bag 1 for each of the packaging bags 1.

Furthermore, when a new continuous packaging bag 3 is introduced into the device main body 11 of the medicine verification device 10 and the measurement unit 19 measures the light transmittance of the packaging bag 1 for the empty bag 1A in the continuous packaging bag 3, the characteristic information acquisition unit 28 acquires a measurement result of the measurement unit 19 every time the measurement is performed.

The update processing unit 29 executes update processing when the verification unit 25 has verified that the type of the verification target medicine matches the type of the medicine appearing in the master image (that is, the type of the medicine specified from the prescription condition information). The update processing is processing for updating the master image as an updating target among the master images registered in the database DB to the image of the medicine having the type verified by the verification unit 25 to match the medicine type appearing in the master image as the updating target (that is, the verification target medicine).

In the update processing, the update processing unit 29 generates update request data and transmits this update request data to the server computer 70. The update request data includes data that specifies the master image as an update target and an image of the verification target medicine to be the updated master image (that is, an image of the verification target medicine verified by the verification unit 25 to match the type of medicine appearing in the master image as an update target). After receiving the update request data from the update processing unit 29, the server computer 70 specifies the master image as an update target from the database DB, and changes the master image to the image of the verification target medicine transmitted from the update processing unit 29. In addition, when the server computer 70 updates the master image, the server computer 70 also updates the information related to the updated master image in the master image management table MT along with the update of the master image.

Furthermore, in the present embodiment, the update processing unit 29 executes the update processing in a case where the verification unit 25 has verified that the type of verification target medicine matches the type of medicine appearing in the master image and a certain condition is satisfied. More specifically, the update processing unit 29 determines whether the verification unit 25 has verified that the type of the verification target medicine matches the type of the medicine appearing in the master image and whether the optical characteristic indicated by the characteristic information acquired by the characteristic information acquisition unit 28 (specifically, the light transmittance of the packaging bag 1 packing the verification target medicine) exceeds a threshold. Subsequently, in a case where the above conditions are satisfied, the update processing unit 29 executes the update processing of setting the captured image of the verification target medicine (that is, the medicine whose type is verified to match the type of the medicine appearing in the master image), as the master image.

The optical characteristic determination unit 30 determines whether the optical characteristic indicated by the characteristic information acquired by the characteristic information acquisition unit 28 (specifically, the light transmittance of the packaging bag 1 packing the verification target medicine) is an optical characteristic that decreases the sharpness of the image of the verification target medicine captured by the image capturing unit 16 to be lower than the sharpness of the master image. Here, the optical characteristics that decreases the sharpness of the image (captured image) of the verification target medicine captured by the image capturing unit 16 to be lower than the sharpness of the master image means an optical characteristic that decreases the sharpness of the captured image to be lower than the sharpness of the master image when the verification target medicine covered by the packaging bag 1 exhibiting the optical characteristic is imaged by the image capturing unit 16.

Specifically, the optical characteristic determination unit 30 specifies the magnitude relationship between the optical characteristic indicated by the characteristic information acquired by the characteristic information acquisition unit 28 and the optical characteristic of the packaging bag 1 packing the medicine appearing in the master image. With this procedure, the optical characteristic determination unit 30 determines whether the optical characteristic indicated by the characteristic information acquired by the characteristic information acquisition unit 28 is an optical characteristic that decreases the sharpness of the captured image of the verification target medicine to be lower than the sharpness of the master image.

More specifically, the optical characteristic determination unit 30 compares the light transmittance indicated by the characteristic information acquired by the characteristic information acquisition unit 28 and the light transmittance of the packaging bag 1 packing the medicine appearing in the master image. Subsequently, when the light transmittance indicated by the characteristic information acquired by the characteristic information acquisition unit 28 is lower, the optical characteristic determination unit 30 determines that the optical characteristic indicated by the characteristic information acquired by the characteristic information acquisition unit 28 is an optical characteristic that decreases the sharpness of the captured image of the verification target medicine to be lower than the sharpness of the master image.

In a case where the characteristic information indicates the light scattering characteristic, such as a haze value, for example, the optical characteristic determination unit 30 compares the haze value indicated by the characteristic information acquired by the characteristic information acquisition unit 28 and the haze value of the packaging bag 1 appearing in the master image. Subsequently, when the haze value indicated by the characteristic information acquired by the characteristic information acquisition unit 28 is larger, the optical characteristic determination unit 30 determines that the optical characteristic indicated by the characteristic information acquired by the characteristic information acquisition unit 28 is an optical characteristic that decreases the sharpness of the captured image of the verification target medicine to be lower than the sharpness of the master image.

As described above, in the present embodiment, it is possible to determine within the processing device 12 using the optical characteristic determination unit 30 as one functional unit of the processing device 12 whether the optical characteristic indicated by the characteristic information acquired by the characteristic information acquisition unit 28 is an optical characteristic that decreases the sharpness of the captured image of the verification target medicine to be lower than the sharpness of the master image. However, the present invention is not limited to this, and the above determination may be performed by a person (for example, an operator of the processing device 12) or another device.

The correction processing unit 31 executes correction processing of correcting the sharpness of the master image in accordance with a determination result obtained by the optical characteristic determination unit 30. The correction processing is image processing that decreases the sharpness of the master image in accordance with the optical characteristic (more precisely, the light transmittance) indicated by the characteristic information acquired by the characteristic information acquisition unit 28. Here, the sharpness of the image is an index indicating the sharpness of the image, indicating clarity of the image (the degree of blurring of the image).

In the present embodiment, the correction processing unit 31 executes correction processing when the optical characteristic determination unit 30 has determined that the optical characteristic indicated by the characteristic information acquired by the characteristic information acquisition unit 28 is an optical characteristic that decreases the sharpness of the captured image of the verification target medicine to be lower than the sharpness of the master image. More specifically, the correction processing unit 31 executes the correction processing when the optical characteristic determination unit 30 has determined that the light transmittance indicated by the characteristic information acquired by the characteristic information acquisition unit 28 (that is, the light transmittance of the packaging bag 1 packing the verification target medicine) is lower than the light transmittance of the packaging bag 1 packing the medicine appearing in the master image.

In the present embodiment, the correction processing unit 31 executes correction processing of blurring the master image in accordance with the light transmittance of the packaging bag 1 packing the verification target medicine, by using an image processing technique for blurring the image. More specifically, the correction processing unit 31 performs Gaussian blurring using a Gaussian function (that is, a Gaussian filter). Specifically, the correction processing unit 31 calculates a spread function corresponding to the light transmittance of the packaging bag 1 packing the verification target medicine and applies this spread function to the master image, thereby blurring the master image.

While the present embodiment blurs the master image by using the Gaussian function (that is, Gaussian filter), the invention is not limited to this. It is also allowable to use other image processing techniques of blurring the image, for example, image processing using a moving average filter (that is, a smoothing filter), or image processing of attenuating high frequency components in an image by using a modulation transfer function.

When the correction processing unit 31 has executed the above-described correction processing, the verification unit 25 verifies the type of the verification target medicine using the master image in which the sharpness has been corrected (that is, the blurred master image).

<<Flow of Medicine Suitability Determination>>

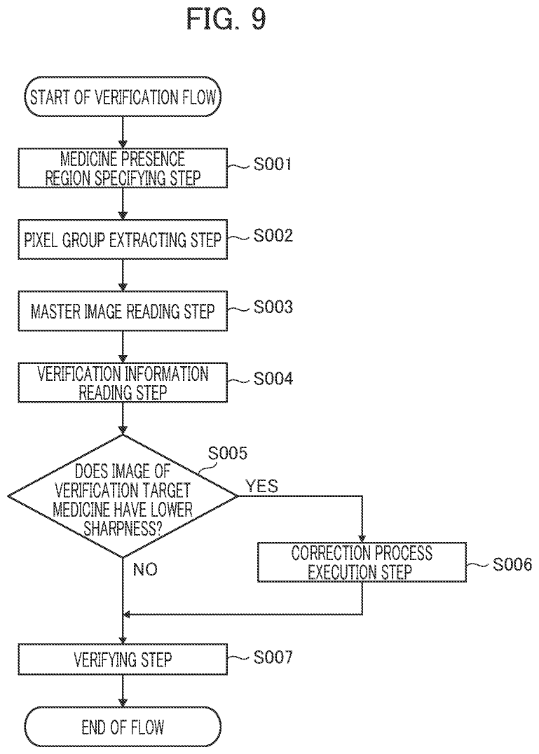

Next, a series of steps (hereinafter, a verification flow) for verifying whether the medicine is properly packaged in accordance with prescription conditions in each of the packaging bags 1 of the continuous packaging bag 3 will be described with reference to FIG. 9. FIG. 9 is a diagram illustrating a general verification flow.

As illustrated in FIG. 9, the verification flow first performs a step of specifying a region including an image of a verification target medicine from the image on which preprocessing has been performed by the preprocessing unit 24 (S001). Hereinafter, an image on which preprocessing has been performed is referred to as a “preprocessed image”, and a region including an image of a medicine in the preprocessed image is referred to as a “medicine presence region”.

In the present embodiment, the preprocessing is performed first, and then a medicine presence region specifying step S001 and a pixel group extracting step S002 described below are performed on the preprocessed image obtained by the preprocessing. However, the present invention is not limited to this mode. It is allowable to use a mode in which the medicine presence region specifying step S001 and the pixel group extracting step S002 are performed on each of the captured images obtained for each of light irradiation directions, and then preprocessing is performed on the captured images for each of irradiation directions in which these steps were performed (more precisely, a medicine extraction image X described below).

The medicine presence region specifying step S001 performs a known edge extraction process and a known segmentation process on the preprocessed image to specify a contour of the medicine in the image. Subsequently, a region surrounded by the specified contour is specified as a medicine presence region. In a case where a plurality of medicines appear in the preprocessed image, the medicine presence regions are specified as many as the number of medicines.

After performing the medicine presence region specifying step S001, the verification unit 25 extracts a pixel group corresponding to the medicine presence region from among the pixel group forming the preprocessed image (S002). As illustrated in FIG. 10, the extracted pixel group forms a rectangle (rectangular region denoted by reference symbol X in FIG. 10) surrounding the medicine presence region. Hereinafter, the extracted pixel group is referred to as a “medicine extraction image X”. In a case where a plurality of medicine presence regions are specified in the medicine presence region specifying step S001, the medicine extraction image X is specified for each of medicine presence regions.

FIG. 10 is a view illustrating the medicine extraction image X. Note that the pixel size (single pixel size with respect to the image) illustrated in FIG. 10 is larger than the actual pixel size for the sake of convenience of illustration.

In the pixel group extracting step S002, the size and the position of the medicine extraction image X are specified. Here, the size of the medicine extraction image X is the area of a rectangular pixel group forming the medicine extraction image X and corresponds to the product of lengths d1 and d2 of the two sides illustrated in FIG. 10.

Furthermore, the position of the medicine extraction image X is a coordinate position when the reference position is an origin and the conveyance direction and the intersecting direction are defined as coordinate axis directions. Specifically, this corresponds to an intersecting position of the diagonal lines of the rectangular pixel group forming the medicine extraction image X, namely the coordinates of point P illustrated in FIG. 10. By specifying the position of the medicine extraction image X in this manner, it is possible to specify the imaging position (arrangement position) of the verification target medicine corresponding to the imaging range of the image capturing unit 16 (more precisely, the angle of view of each of the first camera 16a and the second camera 16b). While the present embodiment is a case where the reference position as the origin is set at the center position of the imaging range of the image capturing unit 16 (more precisely, the first camera 16a and the second camera 16b), the position is not limited to this and it may be set at any position.

After execution of the pixel group extracting step S002, the verification unit 25 executes a step of specifying the type of medicine to be prescribed from the prescription condition information acquired using the prescription condition information acquisition unit 22 and then reading a master image of the specified type of medicine from the database DB stored in the server computer 70 (S003). In this step S003, in a case where a plurality of types of medicines to be prescribed exist, that is, where a plurality of types of medicines are packaged in the packaging bag 1, a master image is read for each of types.

Furthermore, the optical characteristic determination unit 30 accesses the master image management table MT stored in the server computer 70, at a timing when the verification unit 25 reads the master image. Subsequently, the optical characteristic determination unit 30 reads characteristic information corresponding to the master image read by the verification unit 25, specifically, characteristic information indicating the light transmittance of the packaging bag 1 packing the medicine appearing in the master image (S004).

Thereafter, the optical characteristic determination unit 30 specifies the optical characteristic (that is, the light transmittance) of the packaging bag 1 packing the verification target medicine, from the characteristic information acquired by the characteristic information acquisition unit 28. Subsequently, the optical characteristic determination unit 30 determines whether the specified light transmittance is a light transmittance that decreases the sharpness of the captured image of the verification target medicine to be lower than the sharpness of the master image (S005). Specifically, the optical characteristic determination unit 30 compares the light transmittance specified from the specific information acquired by the characteristic information acquisition unit 28 with the light transmittance indicated by the characteristic information read in the previous step S004. At this time, when the light transmittance of the former is lower than the light transmittance of the latter, the optical characteristic determination unit 30 determines that the light transmittance indicated by the characteristic information acquired by the characteristic information acquisition unit 28 is the light transmittance that decreases the sharpness of the captured image of the verification target medicine to be lower than the sharpness of the master image.

Note that step S005 that determines whether the light transmittance of the packaging bag 1 is the light transmittance that decreases the sharpness of the captured image of the verification target medicine to be lower than the sharpness of the master image may be executed by a person or another device instead of the optical characteristic determination unit 30.

Subsequently, when the optical characteristic determination unit 30 has determined that the light transmittance indicated by the characteristic information acquired by the characteristic information acquisition unit 28 is the light transmittance that decreases the sharpness of the captured image of the verification target medicine to be lower than the sharpness of the master image, the correction processing unit 31 executes correction processing on the master image read by the verification unit 25 (S006). In contrast, when the optical characteristic determination unit 30 has determined that the light transmittance indicated by the characteristic information acquired by the characteristic information acquisition unit 28 is light transmittance that increases the sharpness of the captured image of the verification target medicine to be higher than the sharpness of the master image, the process proceeds to next step S007 with no execution of the correction processing by the correction processing unit 31.

Thereafter, the verification unit 25 verifies the type of the verification target medicine and the number of medicines for each of types using the image of the verification target medicine captured by the image capturing unit 16 (more precisely, the medicine extraction image X) and the master image (S007). At this time, in a case where the correction processing unit 31 has executed the correction processing on the master image, the verification unit 25 verifies the type of verification target medicine and the number of medicines for each of types by using the master image in which the sharpness has been corrected (that is, the blurred master image).

In the verification step S007, template matching with the master image is performed for each of the plurality of medicine extraction images X to evaluate the similarity (correlation value) with the master image. Examples of an applicable similarity evaluation method include a known geometric hashing method or a Locally Likely Arrangement Hashing (LLAH) method. Subsequently, it is verified that the type of medicine appearing in the image having the highest similarity among the plurality of medicine extraction images X matches the type of medicine appearing in the master image.

By repeating the above procedure for the read master images (that is, for the number of medicine types indicated by the prescription condition information acquired by the prescription condition information acquisition unit 22), the type is specified for each of verification target medicines. Thereafter, the verification unit 25 totals individually the number of medicines whose types are specified and counts the number of each of types.

The verification is performed by the verification unit 25 using the procedure described above. In addition, when the packaging bag 1 disposed on the arrangement unit 15 is switched (that is, when the verification target medicine changes), the verification is repeated each of times. That is, when the packaging bag 1 disposed on the arrangement unit 15 is switched and an image of the medicine packed in the packaging bag 1 is acquired, verification is performed using the newly acquired image.

When the medicines are packaged under the same prescription condition in each of the packaging bags 1 of the continuous packaging bag 3, the master image used in the first verification can be used as it is in the second and subsequent verifications. Accordingly, step S003 of reading a master image from the database DB may be omitted. Similarly, regarding the characteristic information of the packaging bag 1 appearing in the master image, the characteristic information read before the first verification can be used as it is. Therefore, the characteristic information will not have to be read again in the subsequent verifications.

With the procedure described above, verification is performed on the medicine packed in each of the packaging bags 1 of the continuous packaging bag 3 (more precisely, the packaging bag 1 other than the empty bag 1A), and thus it is possible to inspect whether the medicine is correctly packaged in each of the packaging bags 1 as instructed by the prescription.

<<Basic Operation of Medicine Verification Device>>

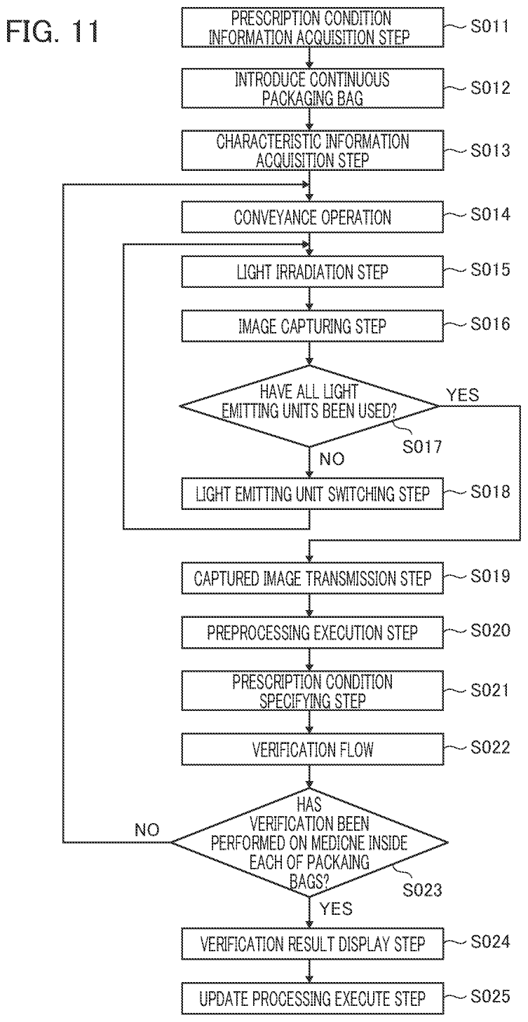

Next, basic operation of the medicine verification device 10 will be described with reference to FIG. 11. FIG. 11 is a diagram illustrating a flow of basic operation of the medicine verification device 10. The medicine verification method of the present invention is implemented in the basic operation of the medicine verification device 10 described below. In particular, a prescription condition information acquisition S011, a characteristic information acquisition step S013, an image capturing step S016, and a verification flow S022 in the basis operation constitute the medicine verification method of the present invention.

First, after completion of the input of the prescription condition information in the prescription input operation, the prescription condition information acquisition unit 22 of the processing device 12 communicates with the prescription condition input device 50 and acquires the prescription condition information indicating the input prescription condition (S011).

Meanwhile, after the automatic packaging operation is performed by the packaging machine 60 in accordance with the input prescription condition (in other words, the prescription condition indicated by the prescription condition information acquired by the prescription condition information acquisition unit 22), the continuous packaging bag 3 having a strip-like shape including the continuously connected packaging bags 1 each of which contains the medicine is created. The continuous packaging bag 3 is introduced into the device main body 11 by the introduction part 13a formed in the housing 13 of the device main body 11 (S012).

The continuous packaging bag 3 introduced into the device main body 11 is moved by the conveyance unit 14 along the conveyance path 18 toward the downstream side in the conveyance direction. At this time, the continuous packaging bag 3 moves in a state where an end (tip) on the side having the empty bag 1A is located on the downstream side in the conveyance direction. When the continuous packaging bag 3 moves to the downstream side in the conveyance direction, the empty bag 1A located on the tip side of the continuous packaging bag 3 passes between the light source 19a and the light receiver 19b of the measurement unit 19 in due course. At this time, the measurement unit 19 measures the light transmittance of the empty bag 1A as characteristic information of the packaging bag 1. Subsequently, after the measurement result of the measurement unit 19 is transmitted to the processing device 12, the characteristic information acquisition unit 28 of the processing device 12 acquires from the measurement unit 19 characteristic information indicating the light transmittance of the packaging bag 1 measured by the measurement unit 19.

Thereafter, the conveyance operation by the conveyance unit 14 is repeated intermittently (S014). With this configuration, after the empty bag 1A of the continuous packaging bag 3 has passed over the arrangement unit 15, the packaging bag 1 adjacent to the empty bag 1A will be disposed on the arrangement unit 15. Subsequently, each time the conveyance operation is performed, the packaging bag 1 in the continuous packaging bag 3, disposed on the arrangement unit 15 is switched. In each of conveyance operations, the continuous packaging bag 3 is conveyed by a predetermined amount to the downstream side in the conveyance direction.

During a period between one conveyance operation and the next conveyance operation (that is, during stoppage of the conveyance of the continuous packaging bag 3), the light irradiation unit 17 emits light to the medicine packed in the packaging bag 1 disposed on the arrangement unit 15 (S015). In this state, using each of cameras, namely, the first camera 16a and the second camera 16b, the image capturing unit 16 captures an image (that is, a verification target medicine) packed in the packaging bag 1 disposed on the arrangement unit 15 (S016).

The light irradiation step S015 by the light irradiation unit 17 will be described in detail. The light irradiation unit 17 emits light from one of the four light emitting units 17a, 17b, 17c, and 17d disposed around the arrangement unit 15, switches the light emitting units sequentially (S017 and S018), and then, emits light again from the newly switched light emitting unit 17a, 17b, 17c, or 17d. That is, the light irradiation unit 17 sequentially switches the light irradiation directions and emits light from each of directions. Subsequently, the image capturing unit 16 captures an image of the verification target medicine for each of light irradiation directions. This enables a total of eight images (the number of cameras×the number of light irradiation directions) to be captured for the medicine packed in the packaging bag 1 disposed on the arrangement unit 15.

The light irradiation step S015, the image capturing step S016, and the light emitting unit switching step S018 described above are repeatedly performed every time the packaging bag 1 disposed on the arrangement unit 15 is switched together with the conveyance operation.

The captured image is transmitted to the image acquisition unit 23 of the processing device 12 as needed (S019). Thereafter, preprocessing is performed by the preprocessing unit 24 of the processing device 12 on the image acquired by the image acquisition unit 23 (S020). This generates a preprocessed image that emphasizes edges of the identification information formed by engraving on the surface of the medicine.

Meanwhile, the verification unit 25 of the processing device 12 specifies a prescription condition for the medicine (that is, the verification target medicine) appearing in the preprocessed image (S021). Specifically, based on the prescription condition information acquired in S011, the verification unit 25 specifies the prescription conditions set for the medicine packaged in each of the packaging bags 1 in the continuous packaging bag 3 (specifically, the type and the number of medicine for each of types).

Thereafter, the verification unit 25 verifies the type and the number of the medicine packaged in each of the packaging bags 1 in the continuous packaging bag 3 following the procedure of the above-described verification flow (S022). In the verification flow, the verification unit 25 accesses the database DB of the server computer 70 and reads the master image corresponding to the prescription condition (specifically, the type of medicine) specified in the previous step S021. Subsequently, the verification unit 25 verifies the type and the number of medicines for each of types packed in the packaging bag 1 disposed on the arrangement unit 15, using the preprocessed image and the master image.

More specifically, the verification unit 25 reads, from the database DB, a master image corresponding to the type of medicine indicated by the prescription information. Furthermore, the optical characteristic determination unit 30 reads, from the master image management table MT, the characteristic information corresponding to the master image read by the verification unit 25 (that is, information indicating the light transmittance of the packaging bag 1 packing the medicine appearing in the master image). Moreover, the optical characteristic determination unit 30 specifies the optical characteristic of the packaging bag 1 disposed on the arrangement unit 15 (that is, the light transmittance of the packaging bag 1 packing the verification target medicine) from the characteristic information acquired by the characteristic information acquisition unit 28 in step S013.

Subsequently, the optical characteristic determination unit 30 specifies the magnitude relationship between the light transmittance of the packaging bag 1 disposed on the arrangement unit 15 and the light transmittance of the packaging bag 1 packing the medicine appearing in the master image. With this procedure, the optical characteristic determination unit 30 determines whether the light transmittance indicated by the characteristic information acquired in step S013 is a light transmittance that decreases the sharpness of the captured image of the verification target to be lower than the sharpness of the master image. When the optical characteristic determination unit 30 has determined that the light transmittance indicated by the characteristic information acquired in step S013 is the light transmittance that decreases the sharpness of the captured image of the verification target to be lower than the sharpness of the master image, the correction processing unit 31 executes correction processing on the master image.

That is, the correction processing unit 31 executes the correction processing in a case where the sharpness of the image, captured by the image capturing unit 16, of the medicine (the verification target medicine) packed in the packaging bag 1 disposed on the arrangement unit 15 is lower than the sharpness of the registered master image stored in the server computer 70. In a case where the correction processing unit 31 has executed the correction processing, the verification unit 25 verifies the type and the number of medicines for each of types packed in the packaging bag 1 disposed on the arrangement unit 15, by using the master image in which the sharpness has been corrected.

On the other hand, in a case where the correction processing unit 31 has not executed the correction processing, the verification unit 25 uses the master image read from the database DB of the server computer 70 as it is and verifies the type and the number of medicines for each of types packed in the packaging bag 1 disposed on the arrangement unit 15.

The above-described series of steps S014 to S022 from the conveyance operation to the verification flow is repeatedly executed every time the packaging bag 1 disposed on the arrangement unit 15 is switched until completion of the verification on the medicines in each of the packaging bags 1 in the continuous packaging bag 3 (S023).

The verification unit 25 performs the above-described verification with the medicine packaged in each of the packaging bags 1 in the continuous packaging bag 3 as a verification target. After completion of all verifications, the verification unit 25 displays character information indicating the result (verification result) on a display (S024). Specifically, the verification unit 25 displays, on the display, character information for notification of the verification result regarding the type of the medicine packed in each of the packaging bags 1 disposed on the arrangement unit 15.

The verification result may be displayed in any manner as long as it is possible to clearly grasp which packaging bag 1 in the continuous packaging bag 3 corresponds to the verification result for the medicine packed in the packaging bag 1. Accordingly, the verification results may be displayed by switching for each of the packaging bags, or verification result for each of the packaging bags 1 constituting the continuous packaging bag 3 may be displayed collectively in association with the position or order of the packaging bag 1.

Thereafter, in a case where the verification unit 25 verifies that the type of the medicine packaged in the packaging bag 1 of the continuous packaging bag 3 matches the type of the medicine appearing in the master image and where the light transmittance of the packaging bag 1 indicated by the characteristic information acquired by the characteristic information acquisition unit 28 exceeds a threshold, the update processing unit 29 executes the update processing (S025). By executing the update processing, the master image as an update target among the master images stored in the server computer 70 is to be updated to a captured image of the verification target medicine (more precisely, the medicine extraction image X) verified by the verification unit 25 to match the type of medicine appearing in the master image.

Thereafter, the conveyance operation of the conveyance unit 14 allows the continuous packaging bag 3 to reach the discharge part of the housing 13 of the device main body 11, and the packaging bag 1 at the end of the continuous packaging bag 3 (the packaging bag 1 located most upstream in the conveyance direction) is discharged to the outside of the housing 13, and the basic operation of the medicine verification device 10 is completed at this point.

The update processing execution step S025 may be performed after the continuous packaging bag 3 is discharged to the outside of the housing 13.

<<Effectiveness of the Medicine Verification Device According to the Present Embodiment>>

As described above, the medicine verification device 10 according to the present embodiment uses the captured image of the verification target medicine (more precisely, the medicine extraction image X) and the master image to verify the type of the verification target medicine. In this verification, the medicine verification device 10 compares the optical characteristics (light transmittance) between the packaging bag 1 packing the verification target medicine and the packaging bag 1 appearing in the master image. Subsequently, in a case where the packaging bag 1 packing the verification target medicine has a lower light transmittance, the medicine verification device 10 executes correction processing of lowering the sharpness of (that is, blurring) the master image. Subsequently, the medicine verification device 10 performs the above verification using the corrected master image.

According to the above configuration, unlike the verification method described in JP 2005-249615 A of simply comparing the captured image of the verification target medicine and the master image, it is possible to calculate the degree of similarity of the images while considering the difference in sharpness between the images. This makes it possible to accurately verify the type of medicine or the like while eliminating the influence of the difference in sharpness between the images on the verification result as much as possible.

Furthermore, the above effect can be further enhanced particularly in a case where master images are frequently updated as described in the verification method in JP 2005-249615 A and where an image of the verification target medicine verified to match the type of medicine appearing in the master image will be registered as a new master image. More specifically, in the case where the master image is updated frequently, the sharpness of the master image (in other words, the light transmittance of the packaging bag 1 appearing in the master image) can change every time the image is updated. In such a case, the sharpness is likely to differ between the captured image of the verification target medicine and the master image, and such a difference in the sharpness might influence the verification result of the type of the medicine.

In contrast, the medicine verification device 10 according to the present embodiment is capable of eliminating the influence of the difference in sharpness between the images on the determination result by the correction processing as described above. Therefore, with the medicine verification device 10 according to the present embodiment, even when the master image is updated, it is difficult to be influenced by the sharpness of the updated master image, leading to accurate verification of the type of medicine or the like.

### Other Embodiments

While the medicine verification device and the medicine verification method of the present invention have been described above with reference to a specific example, the above embodiment is merely an example, and other embodiments are conceivable. For example, while the above embodiment is an exemplary case where a plurality of medicines are packaged in the packaging bag 1 and these medicines (a plurality of medicines) are medicines to be a verification target medicine, the present invention is not limited to this case. The number of medicines packaged in the packaging bag 1 can be set to any number and may be only one, or may be two or more.

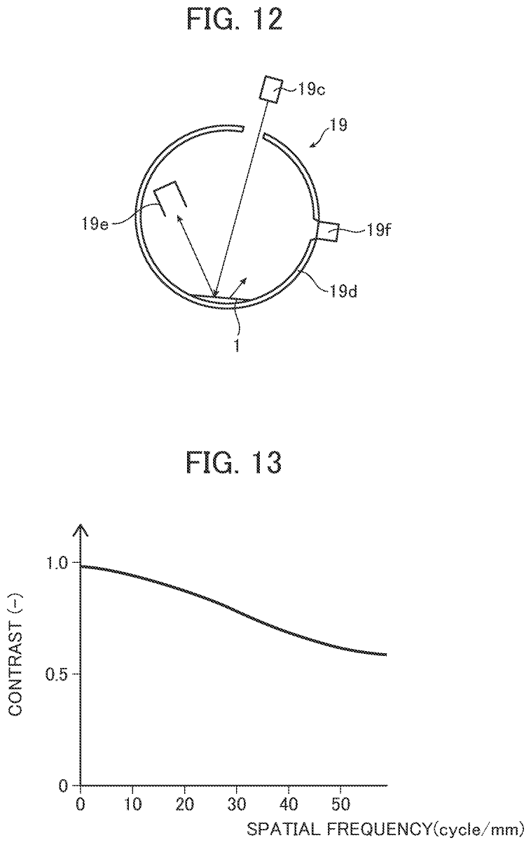

Furthermore, in the above embodiment, the optical characteristic (light transmissivity or light scattering characteristic) of the packaging bag 1 is measured by the measurement unit 19 provided inside the medicine verification device 10. More specifically, in the above embodiment, the measurement unit 19 measures the light transmittance of the packaging bag 1 inside the device main body 11 during conveyance of the continuous packaging bag 3. However, the present invention is not limited to this, and the optical characteristics of the packaging bag 1 may be measured outside the medicine verification device 10 using the measurement unit 19 illustrated in FIG. 12. FIG. 12 is a schematic diagram illustrating a method of measuring the optical characteristics of the packaging bag 1 using an integrating sphere.

The measurement unit 19 illustrated in FIG. 12 is a measuring device disposed outside the medicine verification device 10. The measurement unit 19 includes a light source 19c, a spherical integrating sphere 19d that is hollow and having an opening formed at a position facing the light source 19c, a light shield 19e disposed inside the integrating sphere 19d, and a detector 19f that detects the brightness inside the integrating sphere 19d. In the measurement unit 19 having such a configuration, the packaging bag 1 is disposed inside the integrating sphere 19d, and light is emitted from the light source 19c toward the packaging bag 1 through the opening of the integrating sphere 19d. The light applied onto the packaging bag 1 is emitted from the surface of the packaging bag 1 as scattered light and total reflection light. The total reflection light out of the emitted light is blocked by the light shield 19e. In contrast, the scattered light hits an inner wall surface of the integrating sphere 19d and repeats diffuse reflection. The brightness is eventually uniformized in the integrating sphere 19d so as to be detected by the detector 19f. Subsequently, based on the relationship between the brightness inside the integrating sphere 19d detected by the detector 19f and the amount of light emitted from the light source 19c, the light transmissivity or light scattering characteristics of the packaging bag 1, specifically, the light transmittance or haze value is obtained.

In a case where the light transmissivity or light scattering characteristics of the packaging bag 1 has been measured outside the medicine verification device 10 by using the measurement unit 19 having the above configuration, the characteristic information indicating the measurement result is to be input from the measurement unit 19 to the processing device 12 of the medicine verification device 10. With this configuration, the characteristic information acquisition unit 28 of the processing device 12 acquires the characteristic information indicating the optical characteristics of the packaging bag 1 measured outside the medicine verification device 10.

Furthermore, in the above embodiment, the characteristic information acquisition unit 28 of the processing device 12 acquires the characteristic information indicating light transmissivity such as light transmittance and the light scattering characteristic such as a haze value. However, the characteristic information is not limited to the above information. That is, the characteristic information may be any information as long as it is information indicating optical characteristics that influence the sharpness of the image captured by the image capturing unit 16. For example, the information may be a Modulation Transfer Function curve (hereinafter, the MTF curve) indicating the correspondence relationship between contrast and spatial frequency illustrated in FIG. 13. FIG. 13 is a diagram illustrating an example of the MTF curve. The MTF curve of the packaging bag 1 can be measured using a known measurement method (for example, the square wave chart method).

Note that the characteristic information is preferably information indicating at least one of the light transmittance, the haze value, and the MTF curve of the packaging bag 1, and the information may include information indicating other characteristics (more precisely, optical characteristic that influences the sharpness of the image).

Furthermore, the above-described embodiment has described an exemplary case of the medicine verification device 10 used to inspect the medicines packaged in the packaging bag 1 when the pharmacist prescribes the medicines to patients. However, the use of the medicine verification device 10 is not limited to the above-described use. The medicine verification device 10 may be utilized for the purpose of grasping the type and quantity (more precisely, the quantity of each of types) of the medicines by operators in a facility like a hospital when the medicines are brought by a patient in the packaging bag 1 when admitted to the facility.

What is claimed is:

1. A medicine verification device comprising:
an image capturing unit configured to capture an image of a verification target medicine packed in a packaging material having light transmissivity;
a verification unit configured to verify a type of the verification target medicine using a master image registered in association with the type of medicine and the image of the verification target medicine captured by the image capturing unit;
a characteristic information acquisition unit configured to acquire characteristic information indicating an optical characteristic that influences sharpness of the image captured by the image capturing unit, for the packaging material that packs the verification target medicine; and
a correction processing unit configured to execute correction processing of correcting sharpness of the master image when the optical characteristic indicated by the characteristic information acquired by the characteristic information acquisition unit is an optical characteristic that decreases the sharpness of the image of the verification target medicine captured by the image capturing unit to be lower than the sharpness of the master image,
wherein, when the correction processing unit has executed the correction processing, the verification unit verifies the type of the verification target medicine by using the master image in which the sharpness has been corrected.

2. The medicine verification device according to claim 1, wherein the correction processing unit executes the correction processing of decreasing the sharpness of the master image in accordance with the optical characteristic indicated by the characteristic information acquired by the characteristic information acquisition unit.

3. The medicine verification device according to claim 2, wherein the correction processing unit executes the correction processing of decreasing the sharpness of the master image in accordance with the optical characteristic indicated by the characteristic information acquired by the characteristic information acquisition unit, by using an image processing technique for blurring an image.

4. The medicine verification device according to claim 1, further comprising
a measurement unit configured to measure an optical characteristic of the packaging material,
wherein the measurement unit is provided inside the medicine verification device, and
the characteristic information acquisition unit acquires the characteristic information indicating the optical characteristic measured by the measurement unit.

5. The medicine verification device according to claim 1, further comprising
a measurement unit configured to measure an optical characteristic of the packaging material, the measurement unit being provided outside the medicine verification device,
wherein the characteristic information acquisition unit acquires the characteristic information indicating the optical characteristic measured by the measurement unit by an input of the characteristic information indicating the optical characteristic measured by the measurement unit, from the measurement unit to the medicine verification device.

6. The medicine verification device according to claim 4,
wherein the packaging material is a bag-shaped packaging bag,
the medicine verification device further comprises a conveyance unit configured to convey a strip-shaped continuous packaging bag including continuously arranged packaging bags along a conveyance path,
the image capturing unit captures an image for each of the packaging bags at an intermediate position of the conveyance path, and
the measurement unit measures an optical characteristic for at least one packaging bag of the continuous packaging bag at an intermediate position of the conveyance path.

7. The medicine verification device according to claim 6, further comprising a light irradiation unit configured to emit light toward the packaging bag within an imaging range of the image capturing unit, of the continuous packaging bag, when the image capturing unit captures the image.

8. The medicine verification device according to claim 6,
wherein the continuous packaging bag includes an empty packaging bag containing no medicine, and
the measurement unit measures an optical characteristic of the empty packaging bag.

9. The medicine verification device according to claim 1, wherein the characteristic information acquisition unit acquires the characteristic information indicating at least one of light transmissivity and a light scattering characteristic of the packaging material.

10. The medicine verification device according to claim 9, wherein the characteristic information acquisition unit acquires the characteristic information indicating at least one of light transmittance of the packaging material, a haze value, and a Modulation Transfer Function curve indicating a correspondence relationship between a contrast and a spatial frequency.

11. The medicine verification device according to claim 1, further comprising
a prescription condition acquisition unit configured to acquire prescription condition information indicating prescription conditions set for prescribing a medicine,
wherein the verification unit verifies the type of the verification target medicine using the master image corresponding to the type of the medicine specified by the prescription condition information acquired by the prescription condition acquisition unit and the image of the verification target medicine captured by the image capturing unit.

12. The medicine verification device according to claim 1, further comprising
an optical characteristic determination unit configured to determine whether the optical characteristic indicated by the characteristic information acquired by the characteristic information acquisition unit is an optical characteristic of a case that the sharpness of the image of the verification target medicine captured by the image capturing unit is lower than the sharpness of the master image,
wherein the correction processing unit executes the correction processing when the optical characteristic determination unit determines that the optical characteristic indicated by the characteristic information acquired by the characteristic information acquisition unit is an optical characteristic that decreases the sharpness of the image of the verification target medicine captured by the image capturing unit to be lower than the sharpness of the master image.

13. The medicine verification device according to claim 12, wherein the optical characteristic determination unit specifies a magnitude relationship between the optical characteristic indicated by the characteristic information acquired by the characteristic information acquisition unit and the optical characteristic of the packaging material packing the medicine appearing in the master image, and thereby determines whether the optical characteristic indicated by the characteristic information acquired by the characteristic information acquisition unit is an optical characteristic of a case that the sharpness of the image of the verification target medicine captured by the image capturing unit is decreased to be lower than the sharpness of the master image.

14. A medicine verification method comprising:
a step of capturing, by an image capturing unit, an image of a verification target medicine packed in a packaging material having light transmissivity;
a step of verifying a type of the verification target medicine using a master image registered in association with the type of medicine and an image of the verification target medicine captured by the image capturing unit;
a step of acquiring characteristic information indicating an optical characteristic that influences sharpness of the image captured by the image capturing unit, for the packaging material that packs the verification target medicine; and
a step of executing correction processing of correcting the sharpness of the master image when the optical characteristic indicated by the characteristic information thus acquired is an optical characteristic that decreases the sharpness of the image of the verification target medicine captured by the image capturing unit to be lower than the sharpness of the master image,
wherein, when the correction processing has been executed, the type of the verification target medicine is verified using the master image in which the sharpness has been corrected.

15. The medicine verification method according to claim 14, further comprising a step of determining whether the optical characteristic indicated by the characteristic information thus acquired is an optical characteristic that decreases the sharpness of the image of the verification target medicine captured by the image capturing unit to be lower than the sharpness of the master image.

16. The medicine verification device according to claim 2, further comprising
a measurement unit configured to measure an optical characteristic of the packaging material,
wherein the measurement unit is provided inside the medicine verification device, and
the characteristic information acquisition unit acquires the characteristic information indicating the optical characteristic measured by the measurement unit.

17. The medicine verification device according to claim 3, further comprising
a measurement unit configured to measure an optical characteristic of the packaging material,
wherein the measurement unit is provided inside the medicine verification device, and
the characteristic information acquisition unit acquires the characteristic information indicating the optical characteristic measured by the measurement unit.

18. The medicine verification device according to claim 2, further comprising
a measurement unit configured to measure an optical characteristic of the packaging material, the measurement unit being provided outside the medicine verification device,
wherein the characteristic information acquisition unit acquires the characteristic information indicating the optical characteristic measured by the measurement unit by an input of the characteristic information indicating the optical characteristic measured by the measurement unit, from the measurement unit to the medicine verification device.

19. The medicine verification device according to claim 3, further comprising
a measurement unit configured to measure an optical characteristic of the packaging material, the measurement unit being provided outside the medicine verification device,
wherein the characteristic information acquisition unit acquires the characteristic information indicating the optical characteristic measured by the measurement unit by an input of the characteristic information indicating the optical characteristic measured by the measurement unit, from the measurement unit to the medicine verification device.
